(19) United States
(12) Patent Application Publication
(10) Pub. No.: US 2009/0081801 A1
(43) Pub. Date: Mar. 26, 2009

(54) Process for synthesis of pyrrole derivative, an intermediate for atorvastatin

(76) Inventors: Vinod Kumar Kansal, Faridabad (IN); Brijnath P. Chaurasia, Uttar Pradesh (IN); Hitesh K. Patel, Gujarat (IN); V. Govardhan Rao, Andhra Pradesh (IN); Vaibhav S. Daund, Maharashtra (IN)

Correspondence Address: KENYON & KENYON LLP, ONE BROADWAY, NEW YORK, NY, 10004, US

(21) Appl. No.: 12/228,794

(22) Filed: Aug. 15, 2008

Related U.S. Application Data
(60) Provisional application No. 60/965,042, filed on Aug. 15, 2007.
(60) Provisional application No. 61/010,431, filed on Jan. 7, 2008.

(51) Int. Cl. G01N 33/15; C07D 207/30

(52) U.S. Cl. 436/93; 548/537

(57) ABSTRACT

The present invention also relates to a novel impurity, (6-{2-[2-(6-{2-[2-(4-fluorophenyl)-5-isopropyl-3-phenyl-4-phenylcarbamoyl-pyrrol-1-yl]-ethyl}-2,2-dimethyl-[1,3]-dioxan-4-yl)-acetylamino]-ethyl}-2,2-dimethyl-[1,3]-dioxan-4-yl)-acetic acid tert-butyl ester, the compound of formula IV, having the following structure:

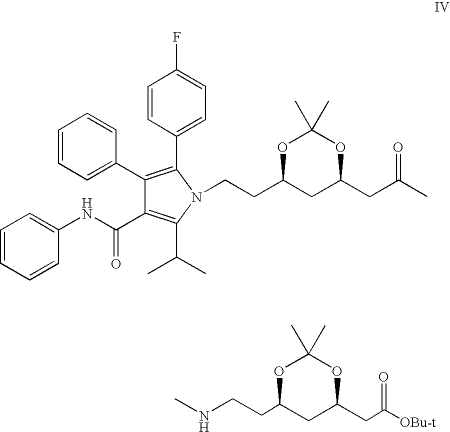

The present invention also provides methods of preparing the compound of formula IV as well as methods of using the compound of formula IV as a reference marker and a reference standard.

The present invention also provides an improved process for preparing (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, the compound of formula I, said process comprising the step of condensing 4-fluoro-α-[2-methyl-1-oxopropyl]-γ-oxo-N,β-diphenylbenzene butanamide, the compound of formula III, with (4R,Cis)-1,1-dimethylethyl-6-(2-aminoethyl)-2,2-dimethyl-1,3-dioxane-4-acetate, the compound of formula II, in the presence of a catalytic amount of an acid in the presence of a solvent system, preferably a binary solvent system, to obtain the desired compound (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester.

### FIELD OF INVENTION

The present invention relates to an improved process for the synthesis of pyrrole derivative i.e. (4R,cis)-6-[2-[3-phenyl-4-(phenyl-carbamoyl)-2-(4-fluorophenyl)-5-(1-methyl-ethyl)-pyrrole-1-yl]-2,2-dimethyl-1,3 dioxane-4-yl-acetic acid-tertiary butyl ester, the compound of formula I, an intermediate for the synthesis of Atorvastatin.

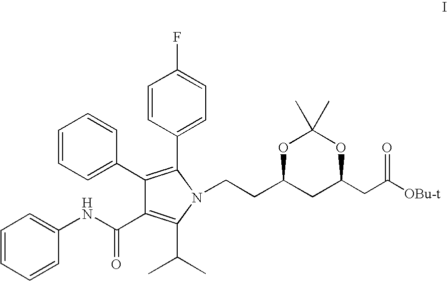

The present invention also relates to a novel impurity, (6-{2-[2-(6-{2-[2-(4-fluorophenyl)-5-isopropyl-3-phenyl-4-phenylcarbamoyl-pyrrol-1-yl]-ethyl}-2,2-dimethyl-[1,3]-dioxan-4-yl)-acetylamino]-ethyl}-2,2-dimethyl-[1,3]-dioxan-4-yl)-acetic acid tert-butyl ester, the compound of formula (IV), having the following structure:

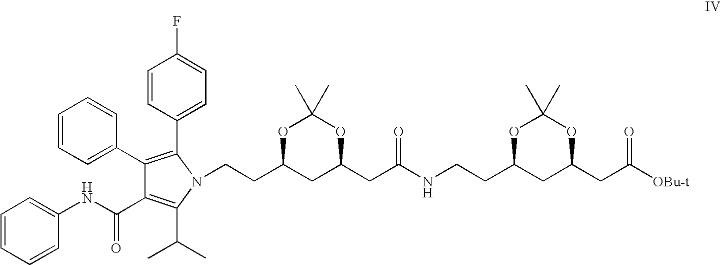

### BACKGROUND OF THE INVENTION

Atorvastatin is a synthetic lipid lowering agent that acts as an inhibitor of 3-hydroxy-3-methylglutaryl-coenzyme A (HMG-CoA). HMG-CoA is a key enzyme in the biosynthesis of cholesterol in humans. Its inhibition leads to a reduction in the rate of biosynthesis of cholesterol. Atorvastatin is indicated for use for reducing elevated total cholesterol, low density lipoprotein cholesterol, apolipoprotein B and high plasma triglycerides in patients with primary hypercholesterolemia and mixed hyperlipidemia. Atorvastatin has the following structure:

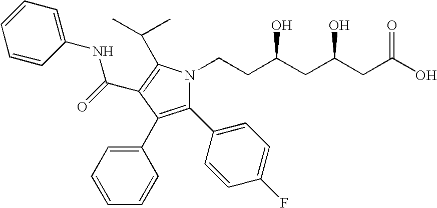

U.S. Pat. Nos. 5,003,080; 5,097,045; 5,149,837; 5,216,174, 5,245,045 and 5,280,126 disclose methods for preparing Atorvastatin free acid and lactone and/or stereoisomers thereof.

U.S. Pat. No. 4,681,893 describes a synthesis of racemic Atorvastatin by utilizing a nine step process inclusive of the synthesis of the heptanoic acid side chain.

U.S. Pat. No. 5,273,995 describes a synthesis of R,R stereoisomer of Atorvastatin.

U.S. Pat. No. 5,298,627 discloses a process for preparing Atorvastatin in one convergent step using a 3,5-heptanoic acid side chain. A precursor of the side chain of Atorvastatin was made by Claisen condensation of N,N-diphenyl acetamide and 4-cyano-3-hydroxybutanoic acid ethyl ester. The resulting 6-cyano-3,5-dihydroxy hexanoic acid amide was protected with 2,2-dimethoxypropane. The nitrile was reduced with Raney nickel and the resulting (4R,Cis)-1,1-dimethylethyl-6-(2-aminoethyl)-2,2-dimethyl-1,3-dioxane-4-acetate amine, the compound of formula II, was reacted with Fluoro-α-[2-methyl-1-oxopropyl]-γ-oxo-N,β-diphenylbenzene butanamide (1,4-diketone), the compound of formula III, according to scheme 1 in 2:2:1 n-heptane:toluene:tetrahydrofuran, a ternary solvent system, in the presence of pivalic acid as a catalyst. The reaction of the amine with 1,4-diketone is known as the Paal-Knorr Pyrrole synthesis. It involves addition of a primary amine to both keto groups of the 1,4-diketone and elimination of two moles of water to form the pyrrole. The intermediate acetonide is hydrolyzed to the carboxylic acid with sodium hydroxide to give Atorvastatin as the sodium salt.

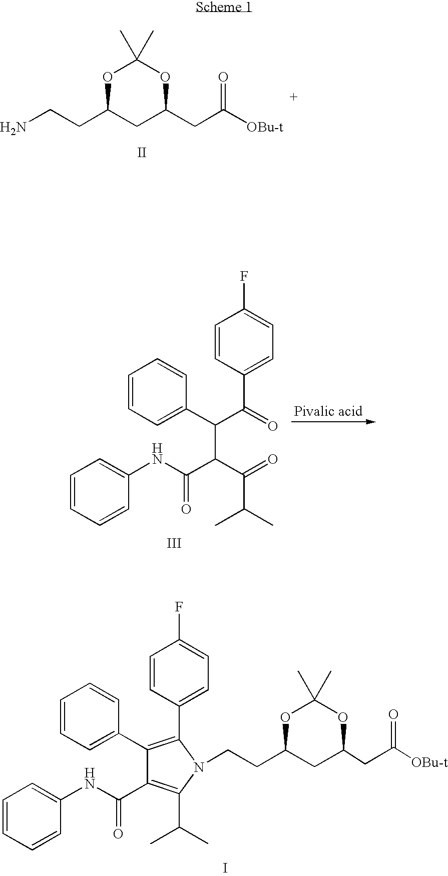

U.S. Pat. No. 5,216,174 teaches generally that the Paal Knorr synthesis can be performed on an acetonide-protected 7-amino-3,5-dihydroxy heptanoic acid tert.-butyl ester in an inert solvent or solvents such as, for example, hexane, toluene and the like for about 24 hours at about reflux temperature of the solvents. The product is not isolated but is treated directly with acid to remove the protecting group.

Baumann, K. L. et al., Tet. Lett. 1992, 33, 2283-2284 described a process for preparing Atorvastatin which involves preparing a pyrrole intermediate in 75% yield from (4R,Cis)-1,1-dimethylethyl-6-(2-aminoethyl)-2,2-dimethyl-1,3-dioxane-4-acetate but does not describe the quality of the condensed product. The Paal Knorr reaction is carried out in a ternary solvent mixture of toluene-heptane-tetrahydrofuran (THF) (1:4:1) in the presence of pivalic acid as catalyst. Another similar condensation between a diketone and amine is described in U.S. Pat. No. 5,397,792 where the condensation is carried out in a 6:10:5 toluene:heptane:THF solvent mixture in the presence of pivalic acid as catalyst.

PCT publication WO 2004/046105 A2 describes a process for the preparation of Atorvastatin by reacting the compound of formula II with the compound of formula III under acidic conditions at elevated temperatures and in a solvent system comprising tetrahydrofuran.

PCT publication WO 2005/118536 describes a process for preparing Atorvastatin by reacting the compound of formula II with the compound of formula III in one or more solvents, including for example hexane, heptane, toluene, tetrahydrofuran, and a mixture thereof, in various combinations in the presence of an acid, for example pivalic acid or p-toluene sulfonic acid.

PCT publication WO 2006/032959 describes a process for preparing Atorvastatin which involves preparing a pyrrole intermediate, reacting the compound of formula II with the compound of formula III in the presence of n-heptanoic acid and/or 2,2-dimethylbutanoic acid in solvents selected from aliphatic hydrocarbons, aromatic hydrocarbons, and mixtures thereof.

IP.COM publication IPCOM000142447D, published Oct. 29, 2006, describes a process for preparing Atorvastatin which involves reacting the compound of formula II with the compound of formula III.

The above prior art documents describe methods for the preparation of Atorvastatin, but do not detail the identity and quantity of the impurities produced by those methods.

### SUMMARY OF THE INVENTION

The present invention provides an improved process for preparing (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, the compound of formula I, said process comprising the step of condensing 4-fluoro-α-[2-methyl-1-oxopropyl]-γ-oxo-N,β-diphenylbenzene butanamide, the compound of formula III, with (4R,Cis)-1,1-dimethylethyl-6-(2-aminoethyl)-2,2-dimethyl-1,3-dioxane-4-acetate, the compound of formula II, in the presence of a catalytic amount of an acid in the presence of a solvent system, preferably a binary solvent system, to obtain the desired compound (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester.

The present invention also relates to a isolated impurity, (6-{2-[2-(6-{2-[2-(4-fluorophenyl)-5-isopropyl-3-phenyl-4-phenylcarbamoyl-pyrrol-1-yl]-ethyl}-2,2-dimethyl-[1,3]-dioxan-4-yl)-acetylamino]-ethyl}-2,2-dimethyl-[1,3]-dioxan-4-yl)-acetic acid tert-butyl ester, of formula IV, having the following structure

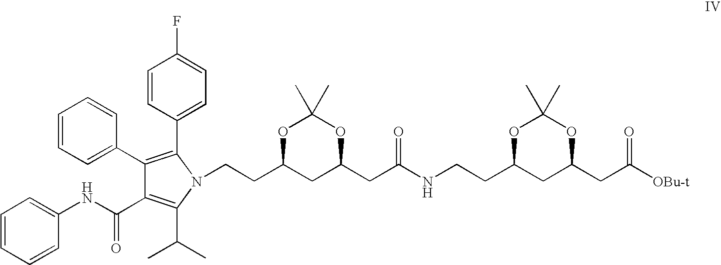

The present invention also relates to an improved process for the preparation of compound of the formula I with compound of formula IV content of less than 0.16%.

The present invention also relates to a process of preparation of the impurity of formula IV, said process comprising of the steps of:
a) combining the compound of formula I in a basic solution and a solvent to obtain a reaction mixture;
b) combining a base with the reaction mixture of step a) with a reactant, to which is added the compound of formula II, to produce the compound of formula IV.

The present invention also provides methods of using the compound of formula IV as a reference marker and a reference standard.

### DETAILED DESCRIPTION OF THE INVENTION

An isolated compound of formula IV

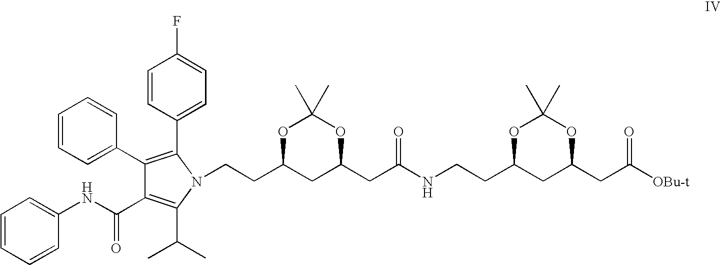

includes a compound of formula IV that has been isolated by column chromatography.

Several prior art publications teach processes for the preparation of the compound of formula I, an intermediate of Atorvastatin. But none of the prior art publications describe the compound of formula IV, which forms en route.

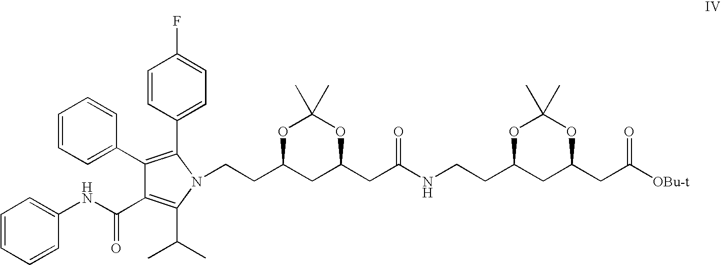

It has now been observed that the compound of formula IV forms by the condensation of the compound of formula I with the compound of formula II and is very difficult to remove. To control the presence of its content in preparations of the compound of formula I, multiple crystallization steps are needed, leading to the loss of the compound of formula I, which decreases the yield of the desired final compound of formula I.

It has surprisingly been found that, by using different solvent systems from those of the prior art in the presence of organic acid to prepare the compound of formula I, a high yield of the compound of formula I with a low level of impurity IV is obtained. Preferably the solvent systems of the present invention are binary solvent systems, including binary solvent systems where one of the solvents is a C₁₋₅ alcohol, preferably methanol. Furthermore, it is not possible to recycle the recovered solvents of the prior art as it is difficult to separate the mixture of ternary solvents. The solvent systems utilized in the present invention enable the production of the intermediate compound of formula I with an amount of impurity of formula IV of not more than 0.16%, thus allowing for a better quality of the final Atorvastatin active pharmaceutical ingredient.

In general, “solvents” in the present invention refers to a broad class of organic and inorganic solvents. The reactions of the present invention may be conducted in a solvent selected from the group consisting of a C₅ to C₁₀ hydrocarbon, a halogenated hydrocarbon, a C₆ to C₁₄ aromatic hydrocarbon, a C₁ to C₅ alcohol, a C₂ to C₇ ester, a C₂ to C₇ ether, a C₁ to C₅ carboxylic acid, water, and suitable mixtures thereof.

Organic solvents may be selected from the group consisting of: a C₆ to C₁₄ aromatic hydrocarbon, a C₁ to C₅ aliphatic hydrocarbon, a C₁ to C₅ alcohol, a C₂ to C₇ ether, a C₁ to C₇ acid, a halogenated hydrocarbon, a C₁ to C₅ organic acid, and suitable mixtures thereof. Organic solvents may also be selected from the group consisting of C₆ to C₁₀ substituted aromatic hydrocarbons, C₁ to C₅ substituted aliphatic hydrocarbons, cyclic ethers, ketones, esters, nitriles, C₄ to C₆ straight, branched or cyclic hydrocarbons, dioxanes, DMF, DMSO, and mixtures thereof.

Halogenated hydrocarbons may include cyclic or acyclic, saturated or unsaturated, aliphatic or aromatic hydrocarbons. Examples of halogenated hydrocarbons include, but are not limited to, halogenated alkanes such as chloromethane, dichloromethane, chloroethane, dichlorotrifluoroethane, difluoroethane, hexachloroethane, and pentafluoroethane; halogenated alkenes such as such as tetrachloroethene, dichloroethene, trichloroethene, vinyl chloride, chloro-1,3-butadiene, chlorotrifluoroethylene; or halogenated benzenes such as benzotrichloride, benzyl chloride, bromobenzene, chlorobenzene, chlorotoluene, dichlorobenzene, fluorobenzene, or trichlorobenzene. The preferred halogen is chlorine. The preferred halogenated hydrocarbons are halogenated aromatic hydrocarbons or halogenated C₁ to C₄ alkanes, and more preferably chlorinated aromatic hydrocarbons or chlorinated C₁ to C₄ alkanes. The more preferred halogenated hydrocarbons are chlorobenzene, o- or p-dichlorobenzene, dichloromethane, or o-chlorotoluene. Preferably, the halogenated hydrocarbon is selected from the group consisting of dichloromethane and dichloroethane.

Aromatic hydrocarbons may be selected from C₅ to C₁₄ aromatic hydrocarbons. Preferable aromatic hydrocarbons are toluene and xylene. Aliphatic cyclic hydrocarbons may be selected from C₃ to C₈ aliphatic cyclic hydrocarbons and preferable aliphatic cyclic hydrocarbons are cyclohexane and cyclopentane. Halogenated hydrocarbons may include cyclic or acyclic, saturated or unsaturated aliphatic or aromatic hydrocarbons. Examples of halogenated hydrocarbons include, but are not limited to, halogenated alkanes such as chloromethane, dichloromethane, chloroethane, dichlorotrifluoroethane, difluoroethane, hexachloroethane, pentafluoroethane; halogenated alkenes such as tetrachloroethene, dichloroethene, trichloroethene, vinyl chloride, chloro-1,3-butadiene, chlorotrifluoroethylene; or halogenated benzenes such as benzotrichloride, benzyl chloride, bromobenzene, chlorobenzene, chlorotoluene, dichlorobenzene, fluorobenzene, or trichlorobenzene. The preferred halogen is chlorine. The preferred halogenated hydrocarbons are halogenated aromatic hydrocarbons or halogenated C₁ to C₄ alkanes, and more preferably chlorinated aromatic hydrocarbons or chlorinated C₁ to C₄ alkanes. The more preferred halogenated hydrocarbons are chlorobenzene, o- or p-dichlorobenzene, dichloromethane, or o-chlorotoluene. Preferably, the halogenated hydrocarbon is selected from the group consisting of dichloromethane and dichloroethane. Ethers may be selected from acyclic or cyclic ethers. Acyclic ethers may include alkyl ethers or arylalkyl ethers wherein the substituents are those described above for the alkyl and arylalkyl groups. For example, acyclic ethers may include diethyl ether, dipropyl ether, isopropyl ether, methyl-tertbutylether, methyl propyl ether, dibutyl ether, ethylene glycol dimethyl ether, dimethoxyethane, bis-methoxymethyl ether and the like. Cyclic ethers may include dioxane, tetrahydrofuran, tetrahydropyran, propylene oxide, phenyloxirane (styrene oxide), cis-2-butene-oxide (2,3-dimethyloxirane), 3-chlorotetrahydrofuran, 2,6-dimethyl-1,4-dioxane, and the like. The preferable ethers are diethyl ether, methyl tertiary butyl ether, and THF.

“Bases” in the present invention refers to a broad group, comprising organic and inorganic bases. Inorganic bases useful in the practice of the present invention are well known to the skilled artisan and include alkali metal carbonates, alkali metal bicarbonates, and alkali metal hydroxides. Organic bases useful in the practice of the present invention include mono-, di- or tri-(C₁ to C₄ alkyl) amines such as N,N-dimethylaniline and N,N-diisopropyl ethyl amine. Tertiary amines are preferred organic bases for use in the practice of the present invention.

The present invention provides an improved process for preparing (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, the compound of formula I, said process comprising the step of condensing 4-fluoro-α-[2-methyl-1-oxopropyl]-γ-oxo-N,β-diphenylbenzene butanamide, the compound of formula III, with (4R,Cis)-1,1-dimethylethyl-6-(2-aminoethyl)-2,2-dimethyl-1,3-dioxan-4-acetate, the compound of formula II, in the presence of a catalytic amount of an acid in the presence of a solvent system, preferably a binary solvent system, to obtain the desired compound (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester.

The present invention also relates to a process of preparing Atorvastatin from the compound of formula I, where the compound of formula I is prepared by the process of the present invention.

The present invention also relates, to a novel impurity, (6-{2-[2-(6-{2-[2-(4-fluorophenyl)-5-isopropyl-3-phenyl-4-phenylcarbamoyl-pyrrol-1-yl]-ethyl}-2,2-dimethyl-[1,3]-dioxan-4-yl)-acetylamino]-ethyl}-2,2-dimethyl-[1,3]-dioxan-4-yl)-acetic acid tert-butyl ester, the compound of formula IV, having the following structure

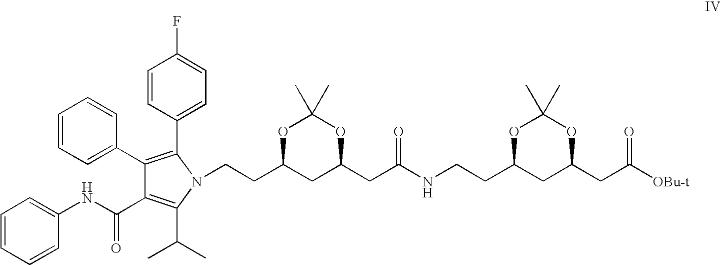

In an embodiment of the present invention, the present invention provides a novel impurity of formula IV, characterized by NMR spectroscopy, having the following characterization data:

¹H-NMR (CDCl₃) 0.82 (m, 1H), 0.93 (t, J=5.94 Hz, 1H), 1.10 (d, J=6.56 Hz, 1H), 1.27 (s, 3H) 1.33 (d, J=2.40 Hz, 6H), 1.40 (s, 1H), 1.49 (d, J=7.08 Hz, 6H), 2.15 (dd, J=4.80 Hz, J=14.8 Hz, 1H), 2.25 (dd, J=6.48 Hz, J=14.8 Hz, 2H), 2.27 (d, J=608 Hz, 1H), 2.38 (dd, J=7.04 Hz, J=14.6 Hz, 1H), 3.24 (m, J=4.27 Hz, 1H), 3.35 (m, J=3.29 Hz, 1H), 3.52 (q, J=7.60 Hz, 1H), 3.66 (t, J=4.28 Hz, 1H), 3.78 (m, J=7.78 Hz, 1H), 3.90 (q, J=3.44 Hz, 1H), 4.03 (m, J=4.58 Hz, 1H), 4.14 (q, J=1.52 Hz, 1H), 4.21 (q, J=3.15 Hz, 1H), 6.30 (t, J=5.05 Hz, 1H), 6.96 (t, J=8.56 Hz, 1H), 7.03 (d, J=7.72 Hz, 1H), 7.13 (d, J=6.80 Hz, 1H)

¹³C-NMR (CDCl₃) δ 19.82, 19.74, 21.57, 21.73, 26.07, 28.09, 30.04, 30.14, 35.34, 35.98, 36.17, 37.99, 42.59, 40.84, 43.38, 36.55, 66.15, 66.17, 80.68, 98.73, 98.81, 115.34, 115.35 (d, J=22.0 Hz), 119.57, 121.79, 123.05, 124.36, 126.56, 128.24 (d, J=3.2 Hz), 128.34, 128.66, 128.76, 128.97, 130.48, 133.17 (d, J=7.9), 134.62, 138.39, 141.44, 162.03 (d, J=247 Hz), 164.80, 170.14, 170.07

The present invention also relates to a process of preparation of the impurity, the compound of formula IV, said process comprising of the steps of:
a) combining the compound of formula I in base and a solvent to obtain a reaction mixture;
b) combining a base with the reaction mixture of step a) with a reactant, to which is added the compound of formula II, to produce the compound of formula IV.

The reactant in the above process is basically meant for acting as a good leaving group or, in other words, an acid activating group, after the formation of an inherent mixed anhydride during the reaction process. The mixed anhydride is not isolated during the reaction. It may be noted that this kind of reaction mechanism (formation of a mixed anhydride in similar types of conditions) is well known among persons skilled in the art. Examples of reactants include ethyl chloroformate and pivalic acid.

In an embodiment of the present invention, the base may be selected from an alcoholic solution of a base. The base may be selected from the group consisting of alkali hydroxides and carbonates. Alkali hydroxides may be selected from the group consisting of sodium hydroxide and potassium hydroxide. Carbonates may be selected from the group consisting of sodium carbonate, sodium bicarbonate, potassium carbonate and potassium bicarbonate. The alcoholic solution of the bases, such as herein described, may be selected from the group consisting of a methanolic solution and an ethanolic solution.

In another embodiment of the present invention, the solvent may be selected from a group such as herein described. In a preferred embodiment, the solvent may be methylene dichloride and/or ethylene dichloride.

In yet another embodiment, the base may be a tertiary amine. In a preferred embodiment, the base may be triethylamine.

In yet another embodiment, the reactant in step b) may be selected from ethyl chloroformate or pivalic acid.

In an embodiment of the present invention, the diamino impurity, the compound of formula IV, may be synthesized by hydrolyzing the compound of formula I in methanolic sodium hydroxide at reflux temperature, followed by adjusting the pH of the reaction mass to 5-6 with acetic acid. The free acid of the compound of formula I may be extracted in methylene dichloride and the organic layer dried over sodium sulphate, followed by addition of triethyl amine at −10 to −5° C. Ethyl chloroformate may be added to the reaction mass slowly at −10 to −5° C. and maintained for 1-2 hours, followed by addition of the compound of formula II. The reaction mass may be maintained for 1-2 hours, followed by washing of the mass with brine solution. The solvent methylene chloride may be stripped off. The residual mass may be subjected to column chromatography to obtain the diamino impurity, the compound of formula IV.

In certain embodiments, the compound of formula IV is obtained in a preparation where more than 50%, more than 75%, more than 85%, more than 95%, more than 97%, more than 98%, or more than 99% of the preparation by weight is the compound of formula IV.

In certain embodiments, the compound of formula IV is obtained in a preparation where about 50%, about 75%, about 85%, about 95%, about 97%, about 98%, or about 99% of the preparation by weight is the compound of formula IV.

In certain embodiments, the compound of formula IV is obtained in a preparation where the preparation contains less than 50%, less than 40%, less than 30%, less than 20%, less than 10%, less than 5%, or less than 1% by weight of any of the compounds of formulas I, II, or III.

In another embodiment, the invention encompasses a process of determining the presence of an impurity in a preparation of Atorvastatin by a process comprising carrying out HPLC with the impurity as a reference standard, wherein the impurity is (6-{2-[2-(6-{2-[2-(4-fluorophenyl)-5-isopropyl-3-phenyl-4-phenylcarbamoyl-pyrrol-1-yl]-ethyl}-2,2-dimethyl-[1,3]-dioxan-4-yl)-acetylamino]-ethyl}-2,2-dimethyl-[1,3]-dioxan-4-yl)-acetic acid tert-butyl ester, the compound of formula IV.

In another embodiment, the invention encompasses a process of determining the presence of an impurity in a preparation of Atorvastatin by a process comprising carrying out HPLC with the impurity as a reference marker, wherein the impurity is (6-{2-[2-(6-{2-[2-(4-fluorophenyl)-5-isopropyl-3-phenyl-4-phenylcarbamoyl-pyrrol-1-yl]-ethyl}-2,2-dimethyl-[1,3]-dioxan-4-yl)-acetylamino]-ethyl}-2,2-dimethyl-[1,3]-dioxan-4-yl)-acetic acid tert-butyl ester, the compound of formula IV.

The present invention also relates to an improved process for the preparation of the compound of formula I, an intermediate for Atorvastatin, with a diamino impurity of formula IV content in a range of 0.05-0.46%, more preferably in a range of 0.05-0.23%. Most preferably, the diamino impurity content is 0.16%.

The present invention provides an improved process for preparing (4R,cis)-6-[2-[3-phenyl-4-(phenyl-carbamoyl)-2-(4-fluorophenyl)-5-(1-methyl-ethyl)-pyrrol-1-yl]-2,2-dimethyl-[1,3]dioxane-4-yl-acetic acid-tertiary butyl ester, the compound of formula I

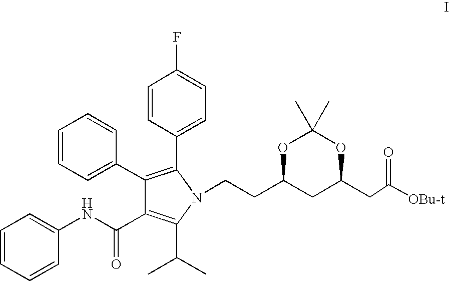

The process comprises condensing 4-fluoro-α-[2-methyl-1-oxopropyl]-γ-oxo-N,β-diphenylbenzene butanamide, the compound of formula III, with (4R,Cis)-1,1-dimethylethyl-6-(2-aminoethyl)-2,2-dimethyl-1,3-dioxane-4-acetate, the compound of formula II, in the presence of a catalytic amount of an acid in the presence of a suitable solvent system, preferably a binary solvent system, in accordance to the scheme given below:

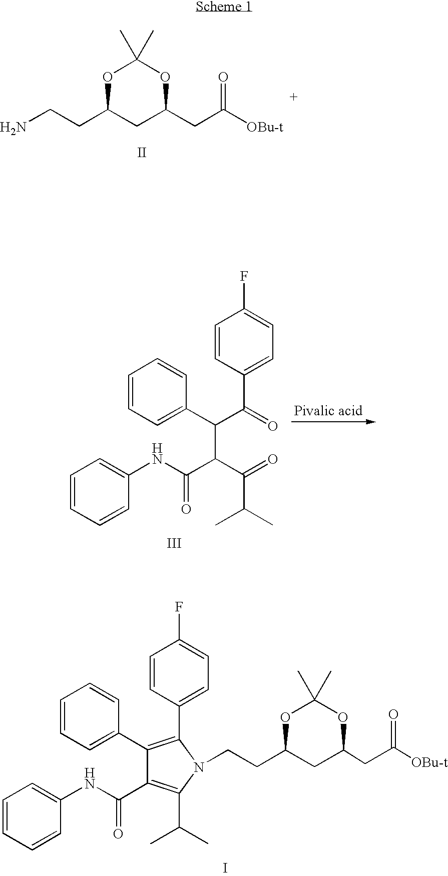

Although the scheme above depicts the use of pivalic acid, it should be understood that the acid may be another organic acid as well.

In one embodiment of the present invention, the organic acid may be selected from the group consisting of pivalic acid, p-toluene sulphonic acid, pyridine hydrobromide, formic acid, methane sulphonic acid, 3-isobutyl glutaric acid, and heptanoic acid.

In a preferred embodiment, the acid may be selected from the group consisting of pivalic acid and heptanoic acid. Preferably, the acid is pivalic acid.

In certain embodiments, the solvent system is a binary solvent system.

In yet another embodiment, the suitable solvent system may be selected from a combination of the groups of hydrocarbons, C₁₋₄ alcohols, halogenated solvents, cyclic ethers and mixtures thereof. In certain embodiments, the solvent system is a binary solvent system where the two solvents are selected from a combination of the groups of hydrocarbons, C₁₋₄ alcohols, halogenated solvents, cyclic ethers and mixtures thereof.

In yet another embodiment, the solvent may be a combination of a non-polar aprotic solvent and a polar protic solvent. In certain embodiments, the solvent system is a binary solvent system where the solvent may be a binary combination of a non-polar aprotic solvent and a polar protic solvent.

Optionally, the non-polar aprotic solvent may be selected from the group consisting of unsubstituted hydrocarbons and substituted hydrocarbons. Preferably, the non-polar aprotic solvent is selected from the group consisting of halogenated hydrocarbons and ethers. More preferably, the halogenated hydrocarbon is ethylene dichloride, methylene dichloride or carbon tetrachloride. The most preferred halogenated hydrocarbon is ethylene dichloride.

Preferably, the unsubstituted hydrocarbons are hexanes or heptanes. Optionally, the ether may be tetrahydrofuran, diisopropyl ether or diethyl ether.

Optionally, the polar protic solvent may be selected from the group consisting of acyclic alcohols, alkyl esters, ketones, and nitriles. Optionally, the acyclic alcohol may be selected from the group consisting of methanol, ethanol, n-butanol, and iso-propyl alcohol. Optionally, the alkyl ester may be ethyl acetate. Optionally, the ketone may be selected from the group consisting of methyl isobutyl ketone and acetone. Optionally, the nitrile may be acetonitrile.

Optionally, a combination of solvents may be selected from the group consisting of methylene chloride and an acyclic alcohol, hexane and ethyl acetate, methylene chloride and hexane, ethyl acetate and heptane, and ethyl acetate and an acyclic alcohol.

In a preferred embodiment, the unsubstituted hydrocarbons may be selected from the group consisting of hexane, cyclohexane, and heptane. The most preferred unsubstituted hydrocarbons is cyclohexane.

In a another embodiment, the C₁₋₄ alcohols may be selected from the group consisting of methanol, ethanol, propanol, isopropanol, and n-butanol. The most preferred C₁₋₄ alcohol is methanol.

In one embodiment of the present invention, halogenated solvents may include halogenated hydrocarbons selected from the group consisting of ethylene dichloride, methylene dichloride, and carbon tetrachloride. The most preferred halogenated hydrocarbon is ethylene dichloride.

The most preferred cyclic ether is tetrahydrofuran.

In one embodiment of the present invention, the mixture of solvents is selected from the group consisting of:

Cyclohexane and an alcohol such as a C₁₋₄ alcohol;
Cyclohexane, an alcohol such as C₁₋₄ alcohol, and a cyclic ether;
Cyclohexane, an alcohol, and a halogenated hydrocarbon.

In yet other embodiment, the solvent mixture comprises:

Toluene:tetrahydrofuran
Cyclohexane:ethyl alcohol
Cyclohexane:isopropyl alcohol
Cyclohexane:t-butyl alcohol
Cyclohexane:methyl alcohol

In a preferred embodiment, the ratio of the solvents in the mixture of solvents is (v/v):

Cyclohexane:alcohol—about 1:0.01 to 1:0.05
Cyclohexane:methyl alcohol:THF—about 15:1:5
Ethylene dichloride:methyl alcohol—about 20:1
Cyclohexane:methyl alcohol:toluene—about 15:5:5
Cyclohexane:toluene:THF:methyl alcohol—about 8:8:4:1

In yet another preferred embodiment, the solvent mixture comprises cyclohexane and an alcohol, such as a C₁₋₄ alcohol. Preferably, cyclohexane forms the major part of the solvent mixture. Preferably, the reaction stops when the alcohol has been used in higher proportion.

Furthermore, the present invention encompasses a method of preparing the compound of formula I comprising condensing 4-fluoro-α-[2-methyl-1-oxopropyl]-γ-oxo-N,β-diphenylbenzene butanamide, the compound of formula III, with (4R,cis)-1,1-dimethylethyl-6-(2-aminoethyl)-2,2-dimethyl-1,3-dioxane-4-acetate, the compound of formula II, in the presence of a catalytic amount of an acid in a suitable solvent system, preferably a binary solvent system, where the yield of the compound of formula I is at least 50% and the level of diamino impurity, the compound of formula IV, is less than 0.47%.

Thus, the present invention provides a method which combines a high yield of the compound of Formula I with a low level of diamino impurity, the compound of formula IV.

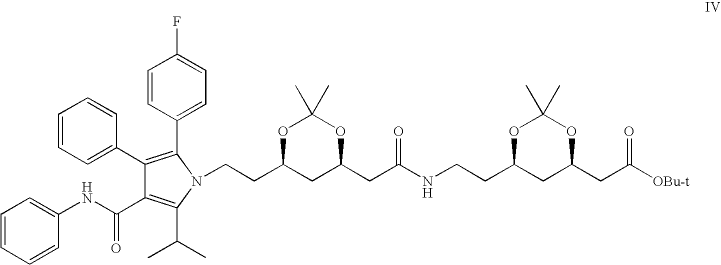

In certain embodiments, the compounds of formula II and III are refluxed in cyclohexane for 60-90 minutes, followed by the addition of the acid and the C₁₋₄ alcohol. In certain embodiments, refluxing for an additional 30-40 hours, preferably about 35 hours, is carried out after the addition of the organic acid and the C₁₋₄ alcohol. In certain embodiments, stirring is carried out between the first and second refluxing, preferably before the addition of the organic acid and the C₁₋₄ alcohol. Water generated during the second refluxing may be removed, preferably azeotropically. Following the second refluxing, the temperature of the reaction may be reduced to about 50-60° C. and the reaction may be washed, preferably with aqueous sodium chloride and/or sodium bicarbonate. Optionally, the compound of formula I may be recovered, e.g., by crystallization, preferably in an ethanol:water mixture.

In certain embodiments, the present invention provides a method for preparing Atorvastatin comprising preparing a compound of formula I comprising condensing 4-fluoro-α-[2-methyl-1-oxopropyl]-γ-oxo-N,β-diphenylbenzene butanamide, the compound of formula III, with (4R, cis)-1,1-dimethylethyl-6-(20aminoethyl)-2,2-dimethyl-1,3-dioxane-4-acetate, the compound of formula II, in the presence of a catalytic amount of an acid in a suitable solvent system, preferably a binary solvent system, and converting the compound of formula I into Atorvastatin. In certain embodiments, the yield of the compound of formula I in the process is at least 50% and the level of diamino impurity, the compound of formula IV, is less than 0.47%.

An important aspect of the present invention is to provide a preparation of the compound of Formula I comprising a level of diamino impurity, the compound of formula IV, of less than 0.47%, less than 0.40%, less than 0.30%, less than 0.25%, or less than 0.20%. In certain embodiments, the preparation of the compound of formula I comprises a level of impurity IV of about 0.46%, about 0.37%, about 0.31%, about 0.29%, about 0.28%, about 0.24%, about 0.22%, about 0.21%, about 0.20%, about 0.18%, or about 0.16%. In certain embodiments, the preparation of the compound of formula I comprises a level of impurity IV of 0.16%-0.18%, 0.16%-0.20%, 0.16%-0.21%, 0.16%-0.22%, 0.16%-0.24%, 0.16%-0.28%, 0.16%-0.29%, 0.16%-0.31%, 0.16%-0.37%, or 0.16%-0.46%.

Another aspect of the present invention is a method of preparation of Atorvastatin, said method comprising converting the compound of formula I into Atorvastatin, where the compound of formula I is present in a preparation comprising a level of diamino impurity, the compound of formula IV, of less than 0.47%, less than 0.40%, less than 0.30%, less than 0.25% and less than 0.20%.

Yet another aspect of the present invention is to provide a method for preparing Atorvastatin comprising converting the compound of formula I into Atorvastatin, where the compound of formula I is present in a preparation comprising a level of diamino impurity, the compound of formula IV, of about 0.46%, about 0.37%, about 0.31%, about 0.29%, about 0.28%, about 0.24%, about 0.22%, about 0.21%, about 0.20%, about 0.18%, or about 0.16%.

Yet another aspect of the present invention is to provide preparations of Atorvastatin comprising low levels of impurity IV. In particular, the present invention provides Atorvastatin preparations comprising a level of diamino impurity, the compound of formula IV, of less than 0.47%, less than 0.40%, less than 0.30%, less than 0.25%, or less than 0.20%. The present invention also provides Atorvastatin preparations comprising a level of impurity IV of about 0.46%, about 0.37%, about 0.31%, about 0.29%, about 0.28%, about 0.24%, about 0.22%, about 0.21% m, about 0.20%, about 0.18%, or about 0.16%. The present invention also provides Atorvastatin preparations comprising a level of impurity IV of 0.16%-0.18%, 0.16%-0.20%, 0.16%-0.21%, 0.16%-0.22%, 0.16%-0.24%, 0.16%-0.28%, 0.16%-0.29%, 0.16%-0.31%, 0.16%-0.37%, or 0.16%-0.46%.

The above stated description of the present invention is illustrated suitably with examples and tables, keeping in context the scope of the invention as disclosed in the specification. The said examples are set forth in order to aid in understanding the invention, but not intended to, and should not be construed to limit its scope in any way.

### HPLC Methodology for the Impurity Determination:

HPLC model: Waters with empower software
Column: Kromasil KR 100 5-C-18, 250 mm×4.6 mm (Akzonobel)
Detector wave length (λ): 254 nm

### Chromatographic Conditions:

Eluent A: Acetonitrile:tetrahydrofuran:Buffer
Eluent B: Acetonitrile:Buffer mixture

### EXAMPLE

### Reference Example

### Example 1

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with toluene (250 ml), 4-Fluoro-α-[2-methyl-1-oxopropyl]-γ-oxo-N,β-diphenylbenzene butanamide (1,4-diketone) (25 g, 0.0599 mole), and (4R,Cis)-1,1-dimethylethyl-6-(2-aminoethyl)-2,2-dimethyl-1,3-dioxane-4-acetate, (21.29 g, 0.0779 mole). The mass was refluxed for 60-90 minutes, followed by stirring at mass temperature of about 90-100° C. Pivalic acid (2.45 g, 0.0239 mole) was added and the reaction mass was refluxed and the water generated in the reaction was removed azeotropically. Reaction mass was maintained at its reflux for 30 hours. Then after mass temperature was brought down to 25-30° C., this was followed by washing with sufficient amount of aq. sodium chloride, aq. sodium bicarbonate, and aq. sodium chloride solution. The solvent was stripped off under reduced pressure and the residue obtained was crystallized from an ethanol:water mixture, filtered and dried to get 18 g (45.92% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 98.38% and a diamino impurity of formula IV of 0.24%.

### Reference Example

### Example 2

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with n-heptane (250 ml), 4-Fluoro-α-[2-methyl-1-oxopropyl]-γ-oxo-N,β-diphenylbenzene butanamide (1,4-diketone) (25 g, 0.0599 mole), and (4R,Cis)-1,1-dimethylethyl-6-(2-aminoethyl)-2,2-dimethyl-1,3-dioxane-4-acetate, (21.29 g, 0.0779 mole). The mass was refluxed for 60-90 minutes, followed by stirring at mass temperature of about 60-70° C. Pivalic acid (2.45 g, 0.0239 mole) was added and the reaction mass was refluxed and the water generated in the reaction was removed azeotropically. Reaction mass was maintained at reflux for 30 hours. Then, after mass temperature was brought down to 60-70° C., this was followed by washing with sufficient amount of aq. sodium chloride, aq. sodium bicarbonate, and aq. sodium chloride solution. The solvent was stripped off under reduced pressure and the residue obtained was crystallized from an ethanol:water mixture, filtered and dried to get 23 g (58.67% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 98.80% and a diamino impurity of formula IV of 0.47%.

### Reference Example

### Example 3

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with THF (500 ml), 4-Fluoro-α-[2-methyl-1-oxopropyl]-γ-oxo-N,β-diphenylbenzene butanamide (1,4-diketone) (50 g, 0.1198 mole), and (4R,Cis)-1,1-dimethylethyl-6-(2-aminoethyl)-2,2-dimethyl-1,3-dioxane-4-acetate, (42.56 g, 0.1557 mole). The mass was refluxed for 60-90 minutes, followed by stirring at mass temperature of about 40-50° C. Pivalic acid (4.89 g, 0.0479 mole) was added and the reaction mass was refluxed for 50 hours. Then the solvent was distilled off under reduced pressure and the residue obtained was crystallized from an ethanol:water mixture, filtered and dried to get 58.48 g (74.6% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 98.77% and a diamino impurity of formula IV of 0.73%.

### Reference Example

### Example 4

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with heptane:toluene:THF (2:2:3) (500 ml), 4-Fluoro-α-[2-methyl-1-oxopropyl]-γ-oxo-N,β-diphenylbenzene butanamide (1,4-diketone) (50 g, 0.1198 mole), and (4R,Cis)-1,1-dimethylethyl-6-(2-aminoethyl)-2,2-dimethyl-1,3-dioxane-4-acetate (42.56 g, 0.1557 mole). The mass was refluxed for 60-90 minutes, followed by stirring at mass temperature of about 40-50° C. Pivalic acid (4.89 g, 0.0479 mole) was added and the reaction mass was refluxed for 35 hours. Then the solvent was distilled off under reduced pressure and the residue obtained was crystallized from an ethanol:water mixture, filtered and dried to give 50 g (63.8% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 97.73% and a diamino impurity of formula IV of 0.54%.

### Reference Example

### Example 5

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with heptane:toluene:THF (2:2:1) (500 ml), 4-Fluoro-α-[2-methyl-1-oxopropyl]-γ-oxo-N,β-diphenylbenzene butanamide (1,4-diketone) (50 g, 0.1198 mole), and (4R,Cis)-1,1-dimethylethyl-6-(2-aminoethyl)-2,2-dimethyl-1,3-dioxane-4-acetate (42.56 g, 0.1557 mole). The mass was refluxed for 60-90 minutes, followed by stirring at mass temperature of about 40-50° C. Pivalic acid (4.89 g, 0.0479 mole) was added and the reaction mass was refluxed for 35 hours. Then the solvent was distilled off under reduced pressure and the residue obtained was crystallized from an ethanol:water mixture, filtered and dried to get 47.04 g (60% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 96.86% and a diamino impurity of formula IV of 0.93%.

### Example 6

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with toluene:THF (3:1) (75 ml), 4-Fluoro-α-[2-methyl-1-oxopropyl]-γ-oxo-N,β-diphenylbenzene butanamide (1,4-diketone) (15 g, 0.0359 mole), and (4R,Cis)-1,1-dimethylethyl-6-(2-aminoethyl)-2,2-dimethyl-1,3-dioxane-4-acetate (10.81 g, 0.0395 mole). The mass was refluxed for 60-90 minutes, followed by stirring at mass temperature of about 40-50° C. Pivalic acid (2.39 g, 0.0233 mole) was added and the reaction mass was refluxed for 24 hours. Then the solvent was distilled off under reduced pressure and the residue obtained was crystallized from an ethanol:water mixture, filtered and dried to get 15.15 g (64.41% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 98.10% and a diamino impurity of formula IV of 0.46%.

### Example 7

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (250 ml), 4-Fluoro-α-[2-methyl-1-oxopropyl]-γ-oxo-N,β-diphenylbenzene butanamide (1,4-diketone) (25 g, 0.0599 mole), and (4R,Cis)-1,1-dimethylethyl-6-(2-aminoethyl)-2,2-dimethyl-1,3-dioxane-4-acetate (21.29 g, 0.0779 mole). The mass was refluxed for 60-90 minutes, followed by stirring at mass temperature of about 40-50° C. Pivalic acid (2.45 g, 0.0024 mole) and ethanol (12.5 ml) were added and the reaction mass was refluxed and the water generated in the reaction was removed azeotropically. Reaction mass was maintained at its reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from an ethanol:water mixture, filtered and dried to get 26 g (66.3% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.3% and a diamino impurity of formula IV of 0.31%.

### Example 8

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (250 ml), 4-Fluoro-α-[2-methyl-1-oxopropyl]-γ-oxo-N,β-diphenylbenzene butanamide (1,4-diketone) (25 g, 0.0599 mole), and (4R,Cis)-1,1-dimethylethyl-6-(2-aminoethyl)-2,2-dimethyl-1,3-dioxane-4-acetate, (21.29 g, 0.0779 mole). The mass was refluxed for 60-90 min. followed by stirring at mass temperature of about 40-50° C. Pivalic acid (2.45 g, 0.0024 mole), isopropyl alcohol (12.5 ml) was added and the reaction mass was refluxed and the water generated in the reaction was removed azeotropically. Reaction mass was maintained at reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from an ethanol:water mixture, filtered and dried to get 24.5 g (62.5% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.47% and a diamino impurity of formula IV of 0.21%.

### Example 9

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (250 ml), 4-Fluoro-α-[2-methyl-1-oxopropyl]-γ-oxo-N,β-diphenylbenzene butanamide (1,4-diketone) (25 g, 0.0599 mole), and (4R,Cis)-1,1-dimethylethyl-6-(2-aminoethyl)-2,2-dimethyl-1,3-dioxane-4-acetate, (21.29 g, 0.0779 mole). The mass was refluxed for 60-90 minutes, followed by stirring at mass temperature of about 40-50° C. Pivalic acid (2.45 g, 0.0024 mole) and t-butyl alcohol (12.5 ml) were added and the reaction mass was refluxed and the water generated in the reaction was removed azeotropically. Reaction mass was maintained at its reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from an ethanol:water mixture, filtered and dried to get 25 g (63.78% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.25% and a diamino impurity of formula IV of 0.29%.

### Example 10

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (500 ml), 4-Fluoro-α-[2-methyl-1-oxopropyl]-γ-oxo-N,β-diphenylbenzene butanamide (1,4-diketone) (50 g, 0.1198 mole), and (4R,Cis)-1,1-dimethylethyl-6-(2-aminoethyl)-2,2-dimethyl-1,3-dioxane-4-acetate, (42.56 g, 0.1157 mole). The mass was refluxed for 60-90 min. followed by stirring at mass temperature of about 40-50° C. Pivalic acid (4.89 g, 0.0439 mole) and methanol (5 ml) were added and the reaction mass was refluxed and the water generated in the reaction was removed azeotropically. Reaction mass was maintained at reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from an ethanol:water mixture, filtered and dried to get 56 g (71.43% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.13% and a diamino impurity of formula IV of 0.28%.

### Example 11

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (500 ml), 4-Fluoro-α-[2-methyl-1-oxopropyl]-γ-oxo-N,β-diphenylbenzene butanamide (1,4-diketone) (50 g, 0.1198 mole), and (4R,Cis)-1,1-dimethylethyl-6-(2-aminoethyl)-2,2-dimethyl-1,3-dioxane-4-acetate (42.56 g, 0.1557 mole). The mass was refluxed for 60-90 minutes, followed by stirring at mass temperature of about 40-50° C. Pivalic acid (4.89 g, 0.0478 mole), methanol (10 ml) was added and the reaction mass was refluxed and the water generated in the reaction was removed azeotropically. Reaction mass was maintained at reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from an ethanol:water mixture, filtered and dried to get 55.2 g (70.41% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.22% and a diamino impurity of formula IV of 0.24%.

### Example 12

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (500 ml), 4-Fluoro-α-[2-methyl-1-oxopropyl]-γ-oxo-N,β-diphenylbenzene butanamide (1,4-diketone) (50 g, 0.1198 mole), and (4R,Cis)-1,1-dimethylethyl-6-(2-aminoethyl)-2,2-dimethyl-1,3-dioxane-4-acetate, (42.56 g, 0.1557 mole). The mass was refluxed for 60-90 minutes, followed by stirring at mass temperature of about 40-50° C. Pivalic acid (4.89 g, 0.0478 mole) and methanol (12.5 ml) were added and the reaction mass was refluxed and the water generated in the reaction was removed azeotropically. Reaction mass was maintained at its reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from an ethanol:water mixture, filtered and dried to get 55.5 g (70.8% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.14% and a diamino impurity of formula IV of 0.22%.

### Example 13

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (500 ml), 4-Fluoro-α-[2-methyl-1-oxopropyl]-γ-oxo-N,β-diphenylbenzene butanamide (1,4-diketone) (50 g, 0.1198 mole), and (4R,Cis)-1,1-dimethylethyl-6-(2-aminoethyl)-2,2-dimethyl-1,3-dioxane-4-acetate, (42.56 g, 0.1557 mole). The mass was refluxed for 60-90 minutes, followed by stirring at mass temperature of about 40-50° C. Pivalic acid (4.89 g, 0.0478 mole) and methanol (15 ml) were added and the reaction mass was refluxed and the water generated in the reaction was removed azeotropically. Reaction mass was maintained at reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from an ethanol:water mixture, filtered and dried to get 58.05 g (74.0% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.46% and a diamino impurity of formula IV of 0.20%.

### Example 14

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (1000 ml), 4-Fluoro-α-[2-methyl-1-oxopropyl]-γ-oxo-N,β-diphenylbenzene butanamide (1,4-diketone) (100 g, 0.239 mole), and (4R,Cis)-1,1-dimethylethyl-6-(2-aminoethyl)-2,2-dimethyl-1,3-dioxane-4-acetate, (85.12 g, 0.311 mole). The mass was refluxed for 60-90 minutes, followed by stirring at mass temperature of about 40-50° C. Pivalic acid (9.78 g, 0.095 mole) and methanol (35 ml) were added and the reaction mass was refluxed and the water generated in the reaction was removed azeotropically. Reaction mass was maintained at its reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from ethanol: water mixture, filtered and dried to give 114.5 g (73.02% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.43% and a diamino impurity of formula IV of 0.18%.

### Example 15

### Preparation of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, (I)

A four neck-flask equipped with a condenser, thermometer pocket, drying tube and mechanical stirrer, was charged with cyclohexane (1000 ml), 4-Fluoro-α-[2-methyl-1-oxopropyl]-γ-oxo-N,β-diphenylbenzene butanamide (1,4-diketone) (100 g, 0.239 mole), and (4R,Cis)-1,1-dimethylethyl-6-(2-aminoethyl)-2,2-dimethyl-1,3-dioxane-4-acetate (85.12 g, 0.311 mole). The mass was refluxed for 60-90 minutes, followed by stirring at mass temperature of about 40-50° C. Pivalic acid (9.78 g, 0.095 mole) and methanol (50 ml) were added and the reaction mass was refluxed and the water generated in the reaction was removed azeotropically. Reaction mass was maintained at its reflux for 35 hours. Then after the mass temperature was brought down to 50-60° C., this was followed by washing with sufficient amount of aq. sodium chloride, aq. sodium bicarbonate, and aq. sodium chloride solution. The solvent was stripped off atmospherically and the residue obtained was crystallized from ethanol: water mixture, filtered and dried to give 116.21 g (74.02% yield) of a solid (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester, with HPLC purity of 99.44% and a diamino impurity of formula IV of 0.16%.

What is claimed is:

1. An isolated compound of formula IV

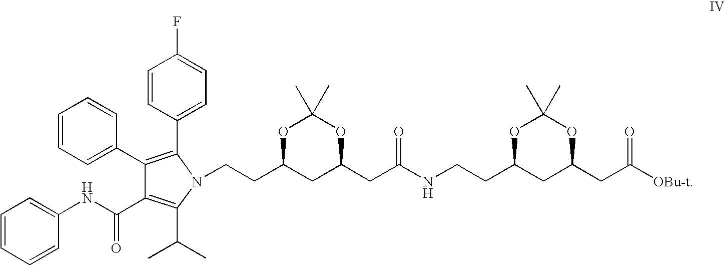

2. A process for preparing a compound of formula IV comprising:
a) combining a compound of formula I in a basic solution and a solvent to obtain a reaction mixture; and
b) combining a base with the reaction mixture of step a) with a reactant, to which is added the compound of formula II, to produce the compound of formula IV.

3. The process of claim 2 where the base is an alkali metal carbonate, an alkali metal bicarbonate, or an alkali metal hydroxide.

4. The process of claim 2 where the base is a mono-, di- or tri-(C₁ to C₄ alkyl) amine.

5. The process of claim 2 where the base is N,N-dimethylaniline, N,N-diisopropyl ethyl amine, or triethylamine.

6. The process of claim 2 where the base is selected from an alcoholic solution of a base.

7. The process of claim 3 where the base is sodium hydroxide, potassium hydroxide, sodium carbonate, sodium bicarbonate, potassium carbonate or potassium bicarbonate.

8. The process of claim 2 where the basic solution is an alcoholic solution of a base.

9. The process of claim 8 where the alcoholic solution of a base is selected from the group consisting of a methanolic solution and an ethanolic solution of a base.

10. The process of claim 8 where the reactant is selected from the group consisting of ethyl chloroformate and pivalic acid.

11. The process of claim 2 for preparing a compound of formula IV comprising:
a) hydrolyzing a compound of formula I in a solution of methanolic sodium hydroxide at reflux temperature,
b) adjusting the pH of the solution of step a) to 5-6,
c) extracting the free acid of the compound of formula I in methylene dichloride,
d) drying the organic layer,
e) cooling the organic layer to −10 to −5° C.,
f) adding triethyl amine to the cooled organic layer,
g) adding ethyl chloroformate to the cooled organic layer to form a reaction mixture,
h) maintaining the reaction mixture for 1-2 hours,
i) adding the compound of formula II to the reaction mixture,
j) maintaining the reaction mixture containing the compound of formula II for 1-2 hours,
j) washing the reaction mixture containing the compound of formula II that has been maintained for 1-2 hours with brine solution,
k) stripping off the methylene chloride to form a residual mass,
l) subjecting the residual mass to column chromatography to obtain the compound of formula IV.

12. A process of determining the presence of an impurity in a preparation of Atorvastatin by a process comprising carrying out HPLC with the impurity as a reference standard, wherein the impurity is (6-{2-[2-(6-{2-[2-(4-fluorophenyl)-5-isopropyl-3-phenyl-4-phenylcarbamoyl-pyrrol-1-yl]-ethyl}-2,2-dimethyl-[1,3]-dioxan-4-yl)-acetylamino]-ethyl}-2,2-dimethyl-[1,3]-dioxan-4-yl)-acetic acid tert-butyl ester, the compound of formula IV.

13. A process of determining the presence of an impurity in a preparation of Atorvastatin by a process comprising carrying out HPLC with the impurity as a reference marker, wherein the impurity is (6-{2-[2-(6-{2-[2-(4-fluorophenyl)-5-isopropyl-3-phenyl-4-phenylcarbamoyl-pyrrol-1-yl]-ethyl}-2,2-dimethyl-[1,3]-dioxan-4-yl)-acetylamino]-ethyl}-2,2-dimethyl-[1,3]-dioxan-4-yl)-acetic acid tert-butyl ester, the compound of formula IV.

14. A process for preparing (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester comprising:
a) condensing 4-fluoro-α-[2-methyl-1-oxopropyl]-γ-oxo-N,β-diphenylbenzene butanamide with (4R,Cis)-1,1-dimethylethyl-6-(2-aminoethyl)-2,2-dimethyl-1,3-dioxane-4-acetate in the presence of a catalytic amount of an acid in a binary solvent system to form (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester; and
b) recovering (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester.

15. The process of claim 14 where the binary solvent system includes solvents selected from the group consisting of a C₆ to C₈ hydrocarbon, a C₂-C₆ halogenated hydrocarbon, a C₆ to C₁₄ aromatic hydrocarbon, a C₁ to C₅ alcohol, a C₄ to C₈ ester, a C₄ to C₈ ether, and mixtures thereof.

16. The process of claim 15 where one of the solvents in the binary solvent system is a C₁ to C₅ alcohol.

17. The process of claim 16 where the C₁ to C₅ alcohol is methanol, ethanol, isopropanol, or t-butanol.

18. The process of claim 17 where the C₁ to C₅ alcohol is methanol.

19. The process of claim 15 where one of the solvents in the binary solvent system is an aliphatic cyclic hydrocarbon.

20. The process of claim 19 where the aliphatic cyclic hydrocarbon is cyclohexane.

21. The process of claim 20 where cyclohexane forms the major part of the binary solvent system.

22. The process of claim 14 where the binary solvent system is a combination of a non-polar aprotic solvent and a polar protic solvent.

23. The process of claim 14 where the acid is selected from the group consisting of pivalic acid, pyridine hydrobromide, formic acid, methane sulphonic acid, 3-isobutyl glutaric acid, and heptanoic acid.

24. The process of claim 23 where the acid is pivalic acid.

25. The process of claim 14 where (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester is recovered with a content of the impurity of formula IV in a range of 0.05-0.46%.

26. The process of claim 25 where (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester is recovered with a content of the impurity of formula IV in a range of 0.05-0.23%.

27. The process of claim 26 where (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester is recovered with a content of the impurity of formula IV of about 0.16%.

28. The process of claim 14 where the yield of (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester is at least 50% and the level of diamino impurity, the compound of formula IV, is less than 0.47%.

29. The process of claim 14 where 4-fluoro-α-[2-methyl-1-oxopropyl]-γ-oxo-N,β-diphenylbenzene butanamide and (4R,Cis)-1,1-dimethylethyl-6-(2-aminoethyl)-2,2-dimethyl-1,3-dioxane-4-acetate are refluxed in cyclohexane for 60-90 minutes.

30. The process of claim 29 where refluxing is followed by the addition of the acid and a C₁₋₄ alcohol.

31. The process of claim 30 where the C₁₋₄ alcohol is methanol, ethanol, isopropanol, or t-butanol.

32. The process of claim 31 where the addition of the acid and a C₁₋₄ alcohol is followed by further refluxing for an additional 30-40 hours.

33. The process of claim 32 where the further refluxing is followed by reducing the temperature of the reaction to about 50-60° C.

34. The process of claim 33 where reducing the temperature of the reaction to about 50-60° C. is followed by washing with aqueous sodium chloride or sodium bicarbonate.

35. The process of claim 34 where, following washing with aqueous sodium chloride or sodium bicarbonate, (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester is recovered by crystallization in an ethanol:water mixture.

36. The process of claim 14 further comprising converting (4R,cis)-6-[2-[3-phenyl-4-(phenyl-carbamoyl)-2-(4-fluorophenyl)-5-(1-methyl-ethyl)-pyrrole-1-yl]-2,2-dimethyl-[1,3]dioxane-4-yl-acetic acid-tertiary butyl ester into Atorvastatin.

37. The process of claim 36 where the Atorvastatin contains less than 0.47% by weight of the compound of formula IV.

38. A process of preparing Atorvastatin from the compound of formula I, prepared by the process such as herein described.

39. The isolated compound of formula IV of claim 1 which has been isolated from the compound of formula I.

40. The isolated compound of formula IV of claim 39 which comprises less than 20% by weight of the compound of formula I.
